(19) United States
(12) Patent Application Publication
(10) Pub. No.: US 2019/0033282 A1
(43) Pub. Date: Jan. 31, 2019

(54) INSPECTION APPARATUS AND INSPECTION SYSTEM

(71) Applicant: KYOCERA Corporation, Kyoto (JP)

(72) Inventor: Hirohide YAMASAKI, Yokohama-shi, Kanagawa (JP)

(73) Assignee: KYOCERA Corporation, Kyoto (JP)

(21) Appl. No.: 16/073,329

(22) PCT Filed: Jan. 16, 2017
(86) PCT No.: PCT/JP2017/001241

§ 371 Date: Jul. 26, 2018

(30) Foreign Application Priority Data
Jan. 27, 2016 (JP) 2016-013566

(51) Int. Cl. G01N 33/02; G01N 31/22; A23B 7/00

(52) U.S. Cl. CPC G01N 33/02; A23B 7/00; G01N 31/22

(57) ABSTRACT

An inspection apparatus includes a container, a detector, and a controller. The container stores a specimen. In accordance with an inspection operation, the detector detects a substance emitted by the specimen stored in the container. The controller judges a quality of the specimen on the basis of the substance detected by the detector.

### CROSS-REFERENCE TO RELATED APPLICATION

The present application claims priority to and the benefit of Japanese Patent Application No. 2016-013566 filed Jan. 27, 2016, the entire contents of which are incorporated herein by reference.

### TECHNICAL FIELD

The present disclosure relates to an inspection apparatus and an inspection system.

### BACKGROUND

A food product management system for managing the deterioration status of a food product is known. For example, patent literature (PTL) 1 discloses a food product management system that collects information related to the deterioration status of a food product stored in a container and manages the deterioration status.

### CITATION LIST

### Patent Literature

PTL 1: JP2005173675A

### SUMMARY

An inspection apparatus according to an embodiment includes a container, a detector, and a controller. The container stores a specimen. In accordance with an inspection operation, the detector detects a substance emitted by the specimen stored in the container. The controller judges a quality of the specimen on the basis of the substance detected by the detector.

An inspection system according to an embodiment includes a detection apparatus and an inspection apparatus. The detection apparatus includes a container, a detector, and a communication interface. The container stores a specimen. The detector detects a substance emitted by the specimen stored in the container. The communication interface transmits information related to the substance detected by the detector to the inspection apparatus. The inspection apparatus includes a communication interface and a controller. The communication interface receives the information related to the substance from the detection apparatus. The controller judges a quality of the specimen on the basis of the received information related to the substance.

### BRIEF DESCRIPTION OF THE DRAWINGS

In the accompanying drawings:

FIG. 1 is an external perspective view schematically illustrating an inspection apparatus according to an embodiment;

FIG. 2 is a functional block diagram illustrating the schematic configuration of the inspection apparatus in FIG. 1;

FIG. 3 is a flowchart illustrating an example of an inspection process executed by the controller in FIG. 2; and

FIG. 4 is a functional block diagram illustrating the schematic configuration of an inspection system according to an embodiment.

### DETAILED DESCRIPTION

The food product management system disclosed in PTL 1 continuously monitors the deterioration status of the food product stored in the container. Therefore, when the user of the food product management system disclosed in PTL 1 wishes to learn the deterioration status of a food product not stored in the container, for example, he cannot do so immediately. The present disclosure can improve the usefulness of an inspection apparatus and an inspection system.

Embodiments are described below in detail with reference to the drawings.

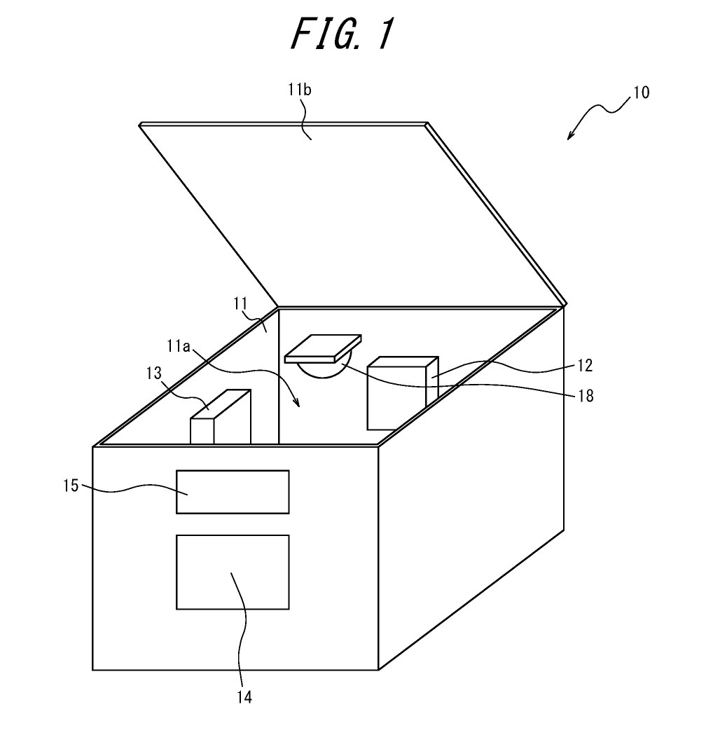

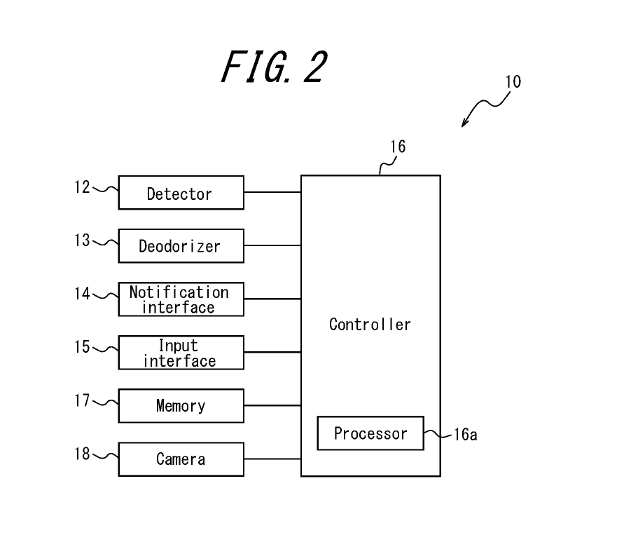

FIG. 1 is an external perspective view schematically illustrating an inspection apparatus according to an embodiment. An inspection apparatus 10 includes a container 11, a detector 12, a deodorizer 13, a notification interface 14, an input interface 15, a controller 16, a memory 17, and a camera 18. Details of the controller 16 and the memory 17 are provided below with reference to the functional block diagram in FIG. 2.

The inspection apparatus 10 executes an inspection process while a specimen is stored in the container 11. In other words, when the inspection apparatus 10 is to execute the inspection process, the user stores the specimen inside the container 11. While the specimen is stored in the container 11, the inspection apparatus 10 uses the detector 12 to detect a substance emitted by the specimen. The inspection apparatus 10 judges the quality of the specimen on the basis of the substance detected by the detector 12. The inspection apparatus 10 executes the inspection process in this way. The notification interface 14 provides notification of the result of the inspection process. An example of the specimen being a food product is described in the embodiment below.

The container 11 is, for example, sealable. The specimen is removably stored in a containment chamber (inspection chamber) 11a of the container 11. The container 11 includes a pivotable lid 11b. The user of the inspection apparatus 10 opens the lid 11b and stores a food product in the containment chamber 11a. The lid 11b need not be pivotable. It suffices for the lid 11b to adopt a state allowing the user to store a food product in the containment chamber 11a. For example, the container 11 may include a lid 11b that is slidable upward or sideways. The container 11 for example includes a tray, on the bottom of the containment chamber 11a, on which the food product can be placed.

The detector 12 detects a substance emitted by the food product that is the specimen. The detector 12 may, for example, be an odor sensor that detects an odor emitted by the food product. The detector 12 may, for example, be a gas sensor that detects a gas emitted by the food product. The detector 12 includes a sensitive membrane and a transducer. The sensitive membrane adsorbs gas molecules that are the source of the odor, and the transducer converts detection of the gas molecules in the sensitive membrane into an electric signal.

The detector 12 may, for example, be provided with a quartz crystal resonator type odor sensor that includes a sensitive membrane, made of an organic thin film, and a quartz crystal resonator. The quartz crystal resonator type odor sensor detects an odor by a change in the resonance frequency of the quartz crystal resonator when gas molecules are adsorbed on the sensitive membrane. The quartz crystal resonator functions as a transducer that converts detection of gas molecules into an electric signal.

The detector 12 may, for example, be provided with a semiconductor gas sensor. The semiconductor gas sensor detects the gas concentration by a change in the resistance of a metal oxide semiconductor when gas molecules are adsorbed on the metal oxide semiconductor. The oxide semiconductor functions as a transducer that converts detection of gas molecules into an electric signal. The detector 12 may, for example, be provided with an infrared absorption spectrum type gas sensor, an electrochemical gas sensor, a contact combustion type gas sensor, a biosensor, or the like.

To adsorb different types of gas molecules, for example, the detector 12 may be provided with a plurality of types of sensitive membranes or metal oxides. The detector 12 may be provided with a plurality of quartz crystal resonator type odor sensors, a plurality of semiconductor gas sensors, or a combination of these sensors. The detector 12 transmits the electric signal converted by the transducer to the below-described controller as odor-related information. The detector 12 is, for example, located inside the container 11. In FIG. 1, the detector 12 is located on an inner side wall of the container 11.

The deodorizer 13 performs a deodorization process on the inside of the container 11. For example, the deodorizer 13 is configured by a degassing apparatus, a deodorizing apparatus, or the like. When the deodorizer 13 is configured to include a degassing apparatus, the deodorizer 13 deodorizes the container 11 by expelling (degassing) gas molecules from inside the container 11 to the outside. When the deodorizer 13 is configured to include an ozone deodorizing apparatus, the deodorizer 13 discharges ozone into the container 11. The discharged ozone decomposes gas molecules to deodorize the inside of the container 11. When the deodorizer 13 is configured to include an oxygen cluster deodorizing apparatus, the deodorizer 13 ionizes the oxygen molecules inside the container 11. The ionized oxygen molecules (oxygen cluster ions) subject gas molecules inside the container 11 to ion decomposition to deodorize the inside of the container 11.

The deodorizer 13 is, for example, located inside the container 11. In FIG. 1, the deodorizer 13 is located on an inner side wall of the container 11.

The notification interface 14 provides notification of the result of the inspection process by the inspection apparatus 10. The notification interface 14 can, for example, provide notification by a visual method using image, character, or color display, light emission, or the like; an auditory method using audio or the like; or a combination of these methods. To provide notification with a visual method, the notification interface 14 may be a display device that provides notification by displaying images or characters. The notification interface 14 may, for example, provide notification by emitting light with an LED or the like. To provide notification with an auditory method, the notification interface 14 may be a speaker or other such sound generating device that provides notification by outputting an alarm, audio guidance, or the like. The notification interface 14 is not limited to providing notification by a visual or auditory method. Any method recognizable by the subject may be adopted. For example, the notification interface 14 may provide notification with a vibration pattern or the like.

In the example in FIG. 1, the notification interface 14 is a display device located outside of the container 11 and provides notification of the result of the inspection process with images, characters, color, or the like.

The input interface 15 accepts operation input from the user and may be configured by operation buttons (operation keys), for example. The input interface 15 may be configured by a touchscreen and accept touch operation input from the user to an input region, displayed on a portion of the display device that is the notification interface 14, for accepting operation input. The user may, for example, instruct the inspection apparatus 10 to start inspection by operating the input interface 15 (by an inspection operation). The user may, for example, input the type of food product that is the specimen by operating the input interface 15.

The inspection apparatus 10 may include the camera 18. On the basis of control by the inspection apparatus 10, the camera 18 captures an image of the specimen. Information related to the imaged specimen is transmitted to the below-described controller along with the odor-related information.

FIG. 2 is a functional block diagram illustrating the schematic configuration of the inspection apparatus 10 in FIG. 1. As illustrated in FIG. 2, the inspection apparatus 10 includes the detector 12, the deodorizer 13, the notification interface 14, the input interface 15, the controller 16, the memory 17, and the camera 18.

The controller 16 is a processor that controls and manages the inspection apparatus 10 overall, starting with the functional blocks of the inspection apparatus 10. The controller 16 is a processor, such as a central processing unit (CPU), that executes a program prescribing control procedures. Such a program is, for example, stored in the memory 17 or on an external storage medium.

To provide control and processing capability for executing various functions, as described below in greater detail, the inspection apparatus 10 is provided with the controller 16, which includes one or more processors 16a.

In various embodiments, the one or more processors 16a may be implemented as a single integrated circuit or as a plurality of communicatively connected integrated circuits and/or discrete circuits. The one or more processors 16a can be implemented with a variety of known techniques.

In an embodiment, the processor 16a includes one or more circuits or units configured to execute one or more data calculation procedures or processes by executing instructions stored in related memory, for example. In another embodiment, the processor 16a may be firmware (such as discrete logic components) configured to execute one or more data calculation procedures or processes.

In various embodiments, the processor 16a may include one or more processors, controllers, microprocessors, microcontrollers, application specific integrated circuits (ASIC), digital signal processors, programmable logic devices, field programmable gate arrays, any combination of these devices or structures, or a combination of other known devices and structures, to execute the below-described functions.

The controller 16 executes the inspection process using the inspection apparatus 10 by controlling the entire inspection apparatus 10. For example, the controller 16 activates the detector 12 on the basis of predetermined operation input that the user of the inspection apparatus 10 provides through the input interface 15. The detector 12 activated by the controller 16 starts to detect an odor emitted by the food product. The controller 16 acquires information related to the odor detected by the detector 12 from the detector 12.

The controller 16 judges the quality of the food product on the basis of the odor detected by the detector 12. In other words, the controller 16 judges the quality of the food product on the basis of the odor-related information acquired from the detector 12. For example, the detector 12 may include a plurality of different odor sensors that adsorb ethylene, ammonia, alcohol, aldehyde, and sulfur gas molecules. The controller 16 may judge the quality of the food product on the basis of a plurality of signals detected by the plurality of odor sensors. The components of gas molecules that are the source of the odor emitted by a food product, the ratio of the components, and the like differ depending on the food product and the quality of the food product. Therefore, the controller 16 judges the quality of a food product on the basis of odor-related information, i.e. the components of gas molecules that are the source of the odor, the ratio of the components, and the like. The controller 16 may, for example, judge the quality of the food product on the basis of the outputs of a plurality of odor sensors and the ratio of the outputs. The controller 16 may, for example, judge the quality of the food product on the basis of features (output value, time constant, or the like) of the response of a plurality of odor sensors. The detector 12 may, for example, include a plurality of ethylene, alcohol, sulfur, and ammonia type odor sensors. When judging the quality of a vegetable, for example, the controller 16 may judge that the quality has deteriorated when the outputs of the alcohol, sulfur, and ammonia type odor sensors among the plurality of odor sensor outputs increase to a predetermined value or higher. The controller 16 refers to information (data), stored in the memory 17, associating the odor emitted by a food product and the quality of the food product to judge the quality of the food product. Here, the quality of a food product refers to the properties or qualities of the food product.

The quality of the food product may, for example, refer to whether the food product is spoiled. In this case, the controller 16 judges whether the food product is spoiled on the basis of the odor-related information. The controller 16 may judge the degree of spoiling of the food product.

The quality of the food product may, for example, refer to whether the food product is ripe. In this case, the controller 16 judges whether the food product is ripe on the basis of the odor-related information. The controller 16 may judge the time at which the food product will be ripe.

The quality of the food product may, for example, refer to the degree of aging of the food product. In this case, the controller 16 judges the degree of aging of a food product, i.e. the degree to which the food product (such as meat) has aged, on the basis of the odor-related information.

The quality of the food product may, for example, refer to the freshness of the food product. In this case, the controller 16 judges the freshness of a food product, i.e. how fresh the food product is, on the basis of the odor-related information.

The quality of the food product may, for example, refer to the production area of the food product. In this case, the controller 16 judges the production area of the food product on the basis of the odor-related information. Specifically, the controller 16 judges whether the food product is domestic or foreign, for example. The controller 16 may judge the specific production area of the food product.

The quality of the food product may, for example, refer to the extent of residual pesticide in the food product. In this case, the controller 16 judges the extent of residual pesticide in the food product, i.e. the amount of residual pesticides in the food product, on the basis of the odor-related information.

The quality of the food product is not limited to the above-described examples and may be any other property or quality related to the food product. The controller 16 may judge the odor using a statistical method, such as principal component analysis, or using a neural network. The controller 16 may generate data by performing a learning process in advance to extract the features of the response of a plurality of odor sensors for each quality of a food product. The controller 16 may store the data after learning in the memory 17. The controller 16 may then judge the quality of the food product on the basis of the degree of matching between the data after learning stored in the memory 17 and the data detected by the detector 12. Based on newly detected data, the controller 16 may update the data after learning stored in the memory 17.

The controller 16 may judge the quality of the food product on the basis of information related to the odor detected by the detector 12 and information related to the specimen imaged by the camera 18. The controller 16 may, for example, infer the type of food product on the basis of the information related to the specimen imaged by the camera 18. A plurality of sets of information (data) associating the odor emitted by a food product and the quality of the food product are stored in the memory 17 for each type of food product. The controller 16 may refer to the information (data) corresponding to the inferred type of food product to judge the quality of the food product.

The controller 16 may judge the quality of the food product on the basis of the odor-related information and the food product type input by operation of the input interface 15. In this case, the controller 16 may refer to the information (data) corresponding to the input type of food product to judge the quality of the food product.

The controller 16 provides notification of information related to the judged quality of the food product through the notification interface 14 as the result of the inspection process. As described above, the notification method may be any method recognizable by the subject. When the notification interface 14 is a display device, as illustrated in FIG. 1, the controller 16 provides notification by display of images, characters, or colors on the display device.

The controller 16 may store information related to the judged quality of the food product in the memory 17 as history data, for example.

Before or after the detector 12 detects an odor, the controller 16 may execute a refresh process to refresh the inside of the container 11. The refresh process is a process to reduce factors that can cause detection errors during odor detection by the detector 12. The controller 16 may, for example, execute the refresh process by activating the deodorizer 13 to degas or deodorize the inside of the container 11. The controller 16 may, for example, execute the refresh process by resetting the detector 12. By being reset, the detector 12 can use the internal state of the container 11 at the time of resetting as a reference to detect a change in the odor inside the container 11 due to an odor emitted by a food product newly stored in the container 11, for example.

The controller 16 can improve the accuracy of odor detection by executing the refresh process before odor detection. The controller 16 can improve the accuracy of odor detection during the next detection process by executing the refresh process after odor detection.

The memory 17 may be configured with a semiconductor memory, a magnetic memory, or the like. The memory 17 stores a variety of information, programs for causing the inspection apparatus 10 to operate, and the like and also functions as a working memory. For example, the memory 17 stores information (data) associating the odor emitted by a food product and the quality of the food product. The memory 17 may, for example, store the result of the detection process by the inspection apparatus 10 as history data.

The camera 18 captures an image of the food product stored in the container 11. The camera 18 transmits information related to the imaged food product to the controller 16. The inspection apparatus 10 may further include an optical emitter to produce a flash when the camera 18 captures an image.

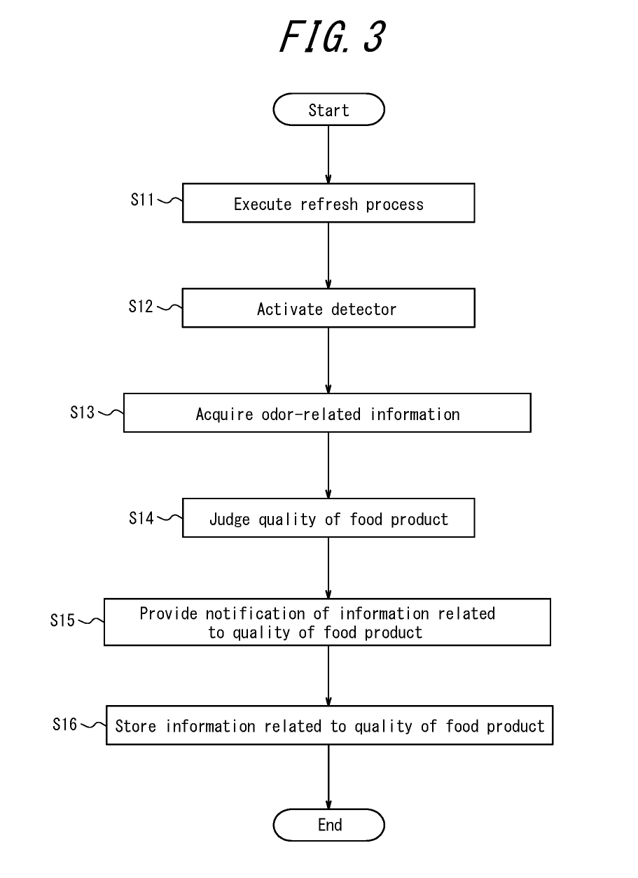

Next, with reference to the flowchart in FIG. 3, an example of the process executed by the controller 16 when the inspection apparatus 10 executes the inspection process is described. Here, the controller 16 is described as executing the refresh process before the odor detection process by the detector 12.

To inspect the quality of a food product using the inspection apparatus 10, the user first opens the lid 11b of the inspection apparatus 10 and places the food product to be inspected in the container 11. The user then closes the lid 11b to seal the container 11. Subsequently, the user instructs the inspection apparatus 10 to start the inspection process by operating the input interface 15 (by an inspection operation).

Once the user provides the predetermined operation input, the controller 16 starts the process in FIG. 3.

The controller 16 executes the refresh process by, for example, activating the deodorizer 13 (step S11).

The controller 16 activates the detector 12 (step S12). By being activated by the controller 16, the detector 12 starts to detect an odor emitted by the food product stored in the container 11.

From the detector 12, the controller 16 acquires the odor-related information obtained by the detector 12 (step S13).

On the basis of the acquired odor-related information, the controller 16 refers to the data stored in the memory 17 to judge the quality of the food product (step S14).

The controller 16 provides notification of information related to the quality of the food product judged in step S14 through the notification interface 14 (step S15). The user can learn the quality of the food product by the notification from the notification interface 14.

The controller 16 stores the information related to the quality of the food product judged in step S14 in the memory 17 (step S16).

Before judging the quality of the food product in step S14, the controller 16 may capture an image of the specimen using the camera 18 and infer the type of food product on the basis of information related to the imaged specimen. In this case, the controller 16 may refer to information (data) corresponding to the inferred type of food product to judge the quality of the food product.

In accordance with an inspection operation, the inspection apparatus 10 according to an embodiment thus uses the detector 12 to detect an odor emitted by a food product stored in the container 11. On the basis of the detected odor, the inspection apparatus 10 judges the quality of the food product. Therefore, when the user wishes to learn the quality of a food product, the user can do so immediately by placing the food product in the container 11 and causing the inspection apparatus 10 to execute the inspection process. Accordingly, the inspection apparatus 10 allows the quality of a food product to be inspected in accordance with user need, thereby improving usefulness as compared to known apparatuses.

The inspection apparatus 10 according to an embodiment allows one inspection apparatus 10 to inspect the quality of a plurality of food products by switching of the food product stored in the container 11.

The inspection apparatus is not limited to the above embodiment, and a variety of modifications and changes may be made. For example, the functions and the like included in the components, steps, and the like may be reordered in any logically consistent way. Furthermore, components, steps, and the like may be combined into one or divided.

For example, the detector 12 has been described in the above embodiment as being an odor sensor that detects an odor emitted by a food product, but the detector 12 may be a sensor other than an odor sensor. For example, the detector 12 may be a gas sensor that detects odorless gas molecules emitted by the specimen.

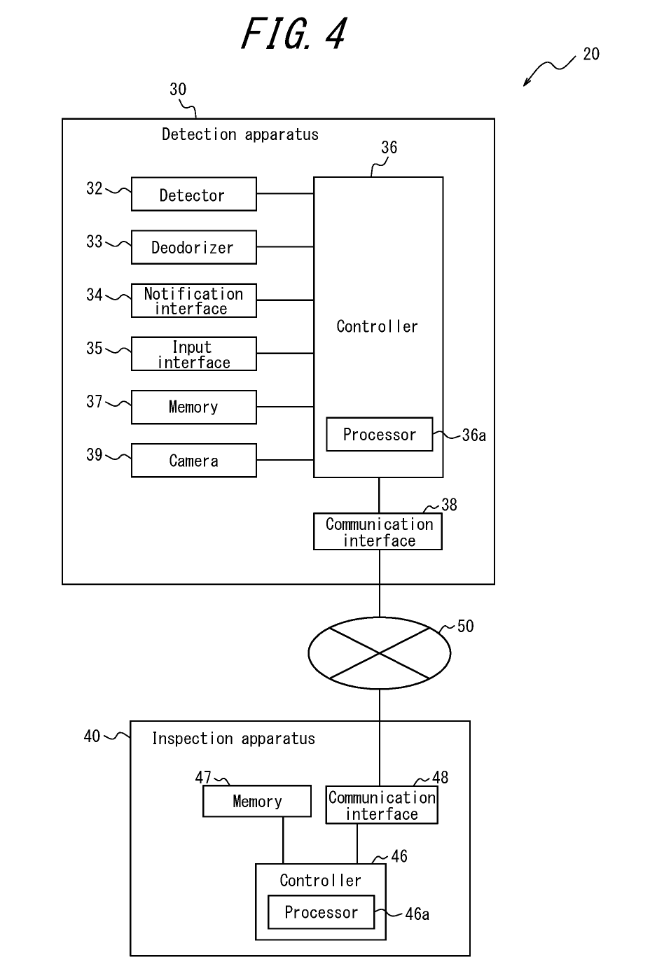

For example, the inspection apparatus 10 according to the above embodiment has been described as including the detector 12 that detects an odor and the controller 16 that judges the quality of a food product. The functional unit that detects an odor and the functional unit that judges the quality, however, may be provided in separate apparatuses capable of communicating with each other. The case of the functional unit that detects an odor and the functional unit that judges the quality being provided in separate apparatuses is described with reference to FIG. 4.

FIG. 4 is a functional block diagram illustrating the schematic configuration of an inspection system according to an embodiment. As illustrated in FIG. 4, the inspection system 20 includes a detection apparatus 30 and an inspection apparatus 40. The detection apparatus 30 and the inspection apparatus 40 connect to allow communication therebetween over a network 50, such as an Internet connection, a wide area network (WAN), or a local area network (LAN).

The detection apparatus 30 may, for example, be configured as a container that includes a lid, as in the inspection apparatus 10 illustrated in FIG. 1. The detection apparatus 30 includes a detector 32, a deodorizer 33, a notification interface 34, an input interface 35, a controller 36, a memory 37, a communication interface 38, and a camera 39. The functions of the detector 32, deodorizer 33, notification interface 34, input interface 35, and camera 39 are respectively similar to the functions of the detector 12, deodorizer 13, notification interface 14, input interface 15, and camera 18 described with reference to FIG. 1. Hence, a description thereof is omitted.

The controller 36 controls and manages the detection apparatus 30 overall, starting with the functional blocks of the detection apparatus 30. To provide control and processing capability for executing various functions, the controller 36 may include at least one processor 36a. The controller 36 does not judge the quality of a food product, unlike the controller 16 described with reference to FIG. 2. The controller 36 transmits odor-related information acquired from the detector 32 to the inspection apparatus 40 through the communication interface 38. The controller 36 may transmit information, related to the imaged specimen, acquired from the camera 39 to the inspection apparatus 40 through the communication interface 38.

From the inspection apparatus 40, the controller 36 acquires information related to the quality of a food product judged by the inspection apparatus 40. Upon acquiring the information related to the quality of the food product, the controller 36 provides notification of the information related to the quality of the food product through the notification interface 34. The controller 36 may store the information related to the quality of the food product in the memory 37.

The communication interface 38 exchanges a variety of information with the inspection apparatus 40 by wired or wireless communication. For example, the communication interface 38 transmits the odor-related information to the inspection apparatus 40. For example, the communication interface 38 receives the information related to the quality of the food product from the inspection apparatus 40. The odor-related information may, for example, be transmitted from the detection apparatus 30 to the inspection apparatus 40 each time the controller 36 acquires the odor-related information from the detector 32 or when the user provides predetermined operation input to the detection apparatus 30. The communication interface 38 may, for example, receive the information related to the imaged specimen from the detection apparatus 30.

The inspection apparatus 40 may, for example, be configured as a server. The inspection apparatus 40 includes a controller 46, a memory 47, and a communication interface 48.

The controller 46 controls and manages the inspection apparatus 40 overall, starting with the functional blocks of the inspection apparatus 40. To provide control and processing capability for executing various functions, the controller 46 may include at least one processor 46a. The controller 46 judges the quality of the food product on the basis of the odor-related information received by the communication interface 48 from the detection apparatus 30. At this time, the controller 46 refers to information (data), stored in the memory 47, associating the odor emitted by a food product and the quality of the food product to judge the quality of the food product. The controller 46 transmits information related to the judged quality of the food product to the detection apparatus 30 through the communication interface 48. The controller 46 may judge the quality of the food product after inferring the type of food product on the basis of the information related to the imaged specimen.

By communicating with the detection apparatus 30 over the network 50, the communication interface 48 transmits and receives a variety of information. For example, the communication interface 48 receives the odor-related information from the detection apparatus 30. For example, the communication interface 48 transmits the information related to the quality of the food product to the detection apparatus 30.

In the inspection system 20 illustrated as an example in FIG. 4, the user can cause the detection apparatus 30 to detect odor-related information by using the detection apparatus 30 with the same method as the method of using the inspection apparatus 10 described with reference to FIGS. 1 and 2. The user can learn the quality of the food product by the notification from the notification interface 34 of the detection apparatus 30.

In particular, since the inspection apparatus 40 that is a server judges the quality of the food product, the data in the memory 47 referred to by the controller 46 of the inspection apparatus 40 can be updated as necessary in the inspection system 20. In other words, the quality of the food product is judged on the basis of updated data in the inspection system 20.

### REFERENCE SIGNS LIST

10, 40 Inspection apparatus

11 Container

11a Containment chamber

11b Lid

12, 32 Detector

13, 33 Deodorizer

14, 34 Notification interface

15, 35 Input interface

16, 36, 46 Controller

16a, 36a, 46a Processor

17, 37, 47 Memory

18, 39 Camera

20 Inspection system

30 Detection apparatus

38, 48 Communication interface

50 Network

1. An inspection apparatus comprising:
a container configured to store a specimen;
a detector configured to detect, in accordance with an inspection operation, a substance emitted by the specimen stored in the container; and
a controller configured to judge a quality of the specimen on the basis of the substance detected by the detector.

2. The inspection apparatus of claim 1, wherein the inspection apparatus executes a refresh process of the container before the detector detects the substance.

3. The inspection apparatus of claim 1, wherein the inspection apparatus executes a refresh process of the container after the detector detects the substance.

4. The inspection apparatus of claim 2, wherein the refresh process includes at least one of degassing the container, deodorizing the container, and resetting the detector.

5. The inspection apparatus of claim 1, further comprising:
a memory configured to store information associating a substance emitted by a specimen and a quality of the specimen; wherein
the controller refers to the information stored in the memory to judge the quality of the specimen stored in the container.

6. The inspection apparatus of claim 1, further comprising a notification interface configured to provide notification of information related to the quality judged by the controller.

7. The inspection apparatus of claim 1, wherein
the specimen is a food product; and
as the quality of the food product, the controller is configured to judge at least one of whether the food product is spoiled, whether the food product is ripe, a degree of aging of the food product, a freshness of the food product, a production area of the food product, and an extent of residual pesticide in the food product.

8. An inspection system comprising:
a detection apparatus; and
an inspection apparatus; wherein
the detection apparatus comprises a container configured to store a specimen, a detector configured to detect a substance emitted by the specimen stored in the container, and a communication interface configured to transmit information related to the substance detected by the detector to the inspection apparatus; and
the inspection apparatus comprises a communication interface configured to receive the information related to the substance from the detection apparatus and a controller configured to judge a quality of the specimen on the basis of the received information related to the substance.
